(19) United States
(12) Patent Application Publication
(10) Pub. No.: US 2023/0131016 A1
(43) Pub. Date: Apr. 27, 2023

(54) METHODS FOR DISEASE ASSESSMENT USING DRAIN FLUID

(71) Applicant: DROPLET BIOSCIENCES, INC., Melrose, MA (US)

(72) Inventors: Jose P. Zevallos, St. Louis, MO (US); Aadel Chaudhuri, Chesterfield, MO (US); Stanley N. Lapidus, Golden, CO (US); Theresa Tribble, Melrose, MA (US)

(21) Appl. No.: 18/087,148

(22) Filed: Dec. 22, 2022

Related U.S. Application Data
(63) Continuation-in-part of application No. 17/947,861, filed on Sep. 19, 2022.
(60) Provisional application No. 63/246,256, filed on Sep. 20, 2021.

(51) Int. Cl. C12Q 1/6886

(52) U.S. Cl. CPC C12Q 1/6886; C12Q 2600/158

(57) ABSTRACT

The present invention provides methods for using surgical drainage waste fluid as a means for diagnosing disease, assessing disease progression, predicting metastatic disease, assessing cancer metastasis, disease staging, molecular staging, and assessing metastatic disease. During surgery, suction is used to drain fluids such as blood, tissue fluids, and other bodily fluids away from the surgery site. The suction drainage fluid waste, also called drain fluid, is removed from the patient during the surgical procedure. Because surgical drain fluid is typically viewed as something that is not useful, it is disregarded and thrown away during the surgery. Instead, the invention provides that drain fluid, which is mostly lymphatic fluid and interstitial fluid, is diagnostically rich and contains important information for assessing, diagnosing, and treating disease. The methods of the invention use this waste fluid for the valuable data it contains. Therefore, while a patient is already undergoing surgery for a medical condition, the waste drain fluid is sampled and analyzed for biomarkers or other molecular indicia of disease.

### FIELD OF INVENTION

The invention generally relates to methods and devices for assessing disease.

### BACKGROUND

Surgical intervention typically results in the expression of drainage fluid or effluent from and around the surgical wound site. The amount of fluid is dependent on, among other things, the pathology being addressed, the location and extent of the surgical intervention, and type of surgery. The drainage fluid typically is removed, either passively or actively, and is regarded as medical waste. Drains are also a common feature of post-operative care and serve to remove fluid build-up from a wound bed. Often, the drains are implanted to aid post-surgical healing and to monitor infection by, for example, assessing the color of the fluid and/or the quantity of fluid being expressed.

Fluid build-up during or after surgery is common and may result from damage to tissue that results in an inflammatory response. In some cases, fluid forms a pocket, or seroma, which can be painful and may become infected. Thus, a common reason for draining fluid either during surgery or post-operatively is to reduce potentially painful swelling and to reduce the risk of painful fluid accumulation due to edema or other post-surgical complications. In addition, a surgeon may clear fluid during a procedure in order to increase access and visibility to tissue at the surgical site.

Other than assessing drain fluid for evidence of infection, which usually involves pus and other detritus from bacterial cells, drain fluid is generally considered waste and is not used for diagnostic purposes.

Pathology is typically performed on samples obtained from a primary lesion (e.g., a solid tumor), form a lymph gland (e.g., a sentinel lymph gland) or from blood. The invention described below provides an alternative source of diagnostic information that leads to greater precision in diagnosis and prognosis.

### SUMMARY

The invention provides methods for using drain fluid as a diagnostic tool. In particular, the invention contemplates various methods for using drain fluid to diagnose cancer, to assess metastasis and for disease prognosis and management. According to the invention, drain fluid contains significant diagnostic and prognostic information. Moreover, assessment of drain fluid as described herein provides greater depth and breadth of diagnosis and prognosis in part because it provides a tool for assessing disease in transit from a primary locus to the lymphatics and on to blood. Accordingly, methods of the invention provide a higher degree of both sensitivity and specificity than would be obtained by assaying a tumor, sentinel lymph gland or blood. As used herein, drain fluid is fluid produced proximal to a tumor during or after a medical intervention.

The invention provides methods for using surgical drainage fluid to track the progression of disease biomarkers over time in a patient. For example, drain fluid is analyzed at a first time point and then analyzed again at one or more subsequent time point(s). Differences in the presence and/or amount of one or more biomarkers in the drain fluid provides information relevant to the diagnosis, staging and prognosis of disease. The drain fluid may essentially be from a single source, such as lymphatic fluid, or may be heterogenous in nature, having contributions from, urine, saliva, cerebrospinal fluid, sputum, interstitial fluid, pleural fluid, bronchioalveolar lavage, and lymph, inflammatory fluids (e.g., fluid resulting from histamine, bradykinin or prostaglandin release), and blood.

In one aspect, methods of the invention are used to assess cancer. Thus, a sample of drain fluid produced proximal to a tumor is obtained at a first point in time and another sample is obtained at a second or subsequent point in time. An accumulation of cancer biomarkers in the drain fluid over time is indicative of possible metastasis. The rate at which biomarker accumulation occurs between samples (the slope of the accumulation curve), as well as the total biomarker accumulation (area under the curve) are diagnostically relevant. In addition, the content of biomarkers in samples at different time points is relevant to diagnosis, staging and prognosis. Thus, according to the invention, the analysis of drain fluid over time provides important information on disease status.

Drain fluid can be obtained at any point in time. However, in certain aspects, the invention provides methods for assessing disease progression in which a first drain fluid sample from a surgical site is obtained at a first time point. A second surgical drainage fluid sample is then obtained from the surgical site at a second time point. In some embodiments, the first fluid sample is obtained during surgery or within 24 hours of a surgery. Disease progression is assessed by identifying a difference in the first sample and the second samples taken at separate time points.

Differences between measurements taken at multiple time points include, but are not limited to, mass (DNA, RNA, Protein) accumulation, biomarker accumulation, rate of change in biomarker amounts, rate of change in the composition of biomarkers, changes in the ratios of biomarkers and/or changes in weighted amounts of biomarkers and/or averages of biomarker amounts. Biomarkers that are useful in the invention include nucleic acids (DNA, RNA, including mRNA, tRNA, rRNA, miRNA), proteins, hormones, receptors (e.g., hormone receptors) and other indicators of disease.

In some embodiments, the biomarker comprises one or more of cells (e.g., tumor cells), an oncogene, interleukin-1, interleukin-6, interleukin-10, a tumor necrosis factor, matrix metalloproteinase-1, matrix metalloproteinase-2, matrix metalloproteinase-9, matrix metalloproteinase-13. In one embodiment, the invention comprises assessing the morphology of cells in samples to determine pathological status. In some instances, cells are stained for identification. In other instances, cells are assessed morphologically. In one aspect, biomarkers for use in the invention are labeled with a detectable label for detection. The samples may be multiplexed across numerous biomarkers or may focus on an individual biomarker, tracking amounts, relative amounts or changes in amounts over time.

In cancer detection, the biomarker can be the presence and/or amount of tumour cells in the sample. An increase in an amount of tumour cells in drain fluid samples taken at different time points is indicative of potential disease progression and/or metastasis.

Methods of the invention may use a difference in the fragment size of cell-free DNA or RNA as a biomarker. An increase in average fragment size between a first fluid sample collected at a first time point, and a second fluid sample collected at a second time point is an indication of disease progression.

In related aspects, the invention comprises identifying a tumor cell obtained from a surgical excision or biopsy and determining the presence of tumor cells in a fluid obtained from a lymphatic. Such methods may be carried out via catheterization of a lymphatic channel. Further, methods of the invention contemplate visualizing the lymphatic channel, using, for example, radiography.

In other aspects, the invention provides methods of diagnosing metastatic disease by identifying a cancer biomarker in lymphatic channel fluid and determining whether the same biomarker is present in a lymph node. Metastatic disease is predicted based on the presence of the biomarker in both the lymphatic channel and the lymph node. In some embodiments, methods include identifying the biomarker in blood.

In a specific embodiment, the invention includes assessing cancer metastasis by identifying circulating tumor cells and/or cell-free tumor DNA in lymphatic fluid at two or more time points. Disease progression is assessed, for example, by comparing the ratio of circulating tumor cells to cell-free DNA in drain fluid samples taken at the two or more time points. A changing (increasing or decreasing) ratio of cell-free DNA to circulating tumor cells over the two or more time points is used to assess the risk of metastasis. In other embodiments, methods include the step of separating the lymphatic fluid from the drain fluid. The lymphatic fluid may be obtained from a surgical site.

In other aspects, the invention provides methods for disease staging wherein a DNA sample is obtained from a sample of tumor tissue and cell-free tumor DNA is identified in the lymphatic fluid, and tumor DNA in blood is identified. Disease stage is assessed based on ratios of tumor DNA in the tumor, lymphatic fluid and blood.

In addition, the invention contemplates staging cancer comprising by determining an amount of cell-free tumor DNA in lymphatic fluid, and an amount of cell-free tumor DNA in blood. Disease stage is assessed based on the relative amounts of cell-free tumor DNA in lymphatic fluid and in blood. Methods may comprise determining a length of the cell-free DNA. In addition, methods include determining a rate of transit of cell-free tumor DNA from the lymph fluid to the blood.

Methods for assessing metastatic disease also include identifying a cancer biomarker in at least two of lymphatic channel fluid, lymph node tissue and blood. Metastatic disease is assessed based on the relative amounts of a biomarker in at least two of the lymphatic channel fluid, lymph node, or blood. The amounts may be weighted.

The invention includes methods for assessing disease by accessing fluid that exists between a tumor and a lymphatic channel, fluid between a lymphatic channel and a lymph node, or fluid between a lymph node and blood and performing an assay on the fluid to detect indicia of disease.

Methods of the invention are also useful to measure biomarker signal in supernatant as opposed to solid components after centrifugation of drain fluid. According to the invention, a supernatant fraction will contain higher concentrations of diagnostic biomarkers, such as immune cells, bacterial cells and the like. The difference between the supernatant fraction and the solid fraction yields additional diagnostic content.

In another aspect, the invention contemplates assessing biomarker information at multiple time points and measuring a rate of decay of biomarker accumulation as an indication of disease regression. In another aspect, the invention comprises measuring biomarkers at multiple time points in response to therapeutic intervention and assessing therapeutic efficacy as an increase in biomarker accumulation in response to therapy as indicative of therapy-induced cell death. According to this aspect of the invention, local measurement of biomarkers is indicative of the effects of systemic therapy. This method can, for example, be used to measure cell-free tumor DNA as an indicator of tumor cell death in response to therapy.

Other aspects and advantages of the invention are apparent to the skilled artisan upon consideration of the following detailed description thereof.

### DETAILED DESCRIPTION

The invention provides methods for using information obtained from drain fluid for assessing disease progression, disease diagnosis, predicting metastatic disease, assessing cancer metastasis, assessing metastatic disease, and disease staging. The invention includes methods for using this what is typically regarded as waste fluid to track the progression of molecular indicia of disease over time in a patient, through migration of tumor cells or cancer biomarkers from tumor to lymphatic channels to lymph nodes to blood.

Methods for assessing disease progression according to the invention include obtaining a first fluid sample from a surgical site at a first time point; obtaining a second sample from the surgical site at a second point; identifying a difference in the first sample and the second sample; and assessing disease progression based on the difference. For example, an increase in the concentration of a biomarker or other quantity of interest between the first sample at the first time point and the second sample at the second time point, is indicative of disease progression.

A first fluid sample may be obtained during a surgical procedure. The surgical procedure may be any surgery, for example, disease or non-disease-related surgical intervention, a biopsy or an excision of a lymph node.

The first fluid sample can be obtained from surgical drain fluid collected during surgery. For example, pumps or suction are used to isolate drain fluids from the surgery site. In some procedures, typically those in which a drain catheter is inserted, the first surgical sample is obtained within 24 hours of the surgery. This fluid sample may contain a mix of fluid types and cellular material, for example, blood, lymphatic fluid, bile, sweat, cerebrospinal fluid, synovial fluid, pleural fluid, peritoneal fluid, saliva, or mucus.

Drain fluid is collected passively or via a catheter, pump, tubing and the like from a surgical site or wound. The drain fluid may be collected by any suitable means, for instance by using a commercially available suction sampling apparatus, such as a Medline specimen sock, designed to attach to an accessory port of a suction canister and connected to suction tubing to safely and in a sterile manner collect a sample from the surgical drainage. Drainage fluid is acceptable if aseptically collected by aspiration into a sterile container after disinfecting the collection tubing. Alternatively, the sample may be collected using a syringe, pipet, or catheter, and transferred to a container for testing or analysis. The container may be any sample vessel, such as a vial, flask, or ampule, suitable for the sterile collection of medical specimens and known to the skilled artisan.

Subsequent samples may be obtained similarly while the patient is undergoing a separate, surgical procedure occurring at a different time point than the first surgical procedure. The collection times are specific points in time wherein the second fluid sample is taken at a later point in time than the first fluid sample. Samples can also be collected via drainage ports inserted into a desired locus in the body of a patient. A minimum of a first and second sample at first and second time points are collected. However, multiple subsequent samples at corresponding multiple subsequent timepoints may also be collected.

Methods of the invention comprise identifying a difference, or differences, between the first fluid sample and the second fluid sample and assessing disease progression, severity, staging, prognosis or diagnosis based on the difference or differences.

Identified differences include presence or absence of one or more biomarkers, changes in quantity, amount, weighted amount, quality, heterogeneity (both in terms of genomic heterogeneity and morphologic heterogeneity), velocity of change, and/or accumulated changes over time between two or more measurements. For example, differences may include the rate of accumulation of a biomarker, an amount of tumor cells, or an amount of cell-free DNA or RNA. The difference may also be the fragment size of cell-free DNA or RNA wherein a decrease in average fragment size between the first fluid sample at the first time point and the second fluid sample at a second time points is indicative of disease progression. The difference may also be measured as presence/absence or positive/negative for the presences of a biomarker or set of biomarkers.

If a biomarker or other quantity of interest has increased in concentration between the first sample at the first time point, and the second sample at the second time point, a difference is identified which is an indication of disease progression or advancement. For example, in cancer, disease progression is often defined by cancer that continues to grow or spread. Progression-free survival (PFS) for patients with cancer is the length of time during and after treatment of a disease that a patient lives with the disease while the disease does not worsen. For clinical trials, measuring the progression-free survival (PFS) is one way to see how well a new treatment works. Therefore, the information obtained from the difference identified between the first fluid sample and the second fluid sample may be used to assess disease progression and inform treatment of disease. Thus, methods of the invention are useful to identify effective therapeutics and to assess the efficacy of treatments. In one embodiment, therapeutic selection and/or efficacy is performed ex vivo in a drain fluid sample prior to administration to a patient. For example, the ex vivo sample may be a tumor biopsy sample. Generally, biomarkers are analyzed in the drain fluid sample to select a putative therapeutic, which is then tested in the ex vivo sample to determine its potential efficacy. In a preferred embodiment, multiple putative therapeutics are analyzed in a multiplexed assay across a number of tumor biopsy samples. Tumor biopsy samples can be cultured in any useful medium. Therapeutic efficacy is also tested in vivo by analyzing drain fluid to determine a putative therapeutic, administering the putative therapeutic and obtaining a second sample to determine the efficacy of the therapeutic. The process may be repeated to determine an effective therapy.

In one embodiment, methods of the invention assess a rate of accumulation of a biological marker (biomarker) as indicative of disease status or progression. Biomarkers are biological molecules found in blood, other body fluids, or tissues that are a sign of a normal or abnormal process, or of a condition or disease. A biomarker may be used to monitor treatment response as well as for diagnosis and prognosis. Cancer biomarkers are biological molecules produced in response to or coincident with cancer. Biomarkers can be DNA, RNA, protein or metabolomic profiles that are specific to the tumor. Testing can include genomic testing of DNA or RNA and may include assessment of fusions, loss of heterozygosity, point mutations, rearrangements, deletions or other alterations in sequence or secondary structure. Biomarkers can be used to assess an individual's risk of developing cancer, or to determine a patient's risk of cancer recurrence. Additionally, biomarkers can be used to predict the likelihood that a given therapy will work for a specific patient, and to monitor a disease's progression to determine if therapy is working.

The rate of accumulation of the biomarker may be represented as the gradual acquisition of a mass or quantity over time, or the progressive increase in concentration over time. In assessing disease progression, the rate of accumulation may offer information about the effect of therapeutic intervention. In addition, the rate of accumulation may identify different tumor types that are amenable to certain therapies and may also identify patients who will benefit from such treatment. The concentration, mass or quantity of a biomarker in the first fluid sample and again in at least a second fluid sample is measured. The difference in concentration, mass or quantity of a biomarker or plurality of biomarkers over time is used to calculate the rate of accumulation of the biomarker. The movement of the biomarker from tumor to lymph channel to lymph node to blood over time, as well as the velocity of that movement and the total accumulation over time may be calculated.

The biomarker can be measured using any suitable method for example sequencing, optical density, probe hybridization, optical morphology, or protein-based assays such as ELISA (enzyme-linked immunosorbent assay).

Biomarkers useful in the invention vary and are selected based on the disease indication being monitored and other factors known to the skilled artisan. Moreover, sensitivity and specificity may vary across biomarkers and that will influence biomarker selection.

Methods of the invention also provide for assessing disease progression by identifying the difference in amounts of tumor cells in first and second drain fluid samples. The exact nature of the cell being measured may vary. For example, tumor cells may be, for example, circulating tumor cells (CTCs), tumor-derived exosomes, or circulating tumor nucleic acids. The invention also contemplates detecting circulating tumor nucleic acids released from tumor cells. Analyzing differences in the quantity and/or amount of tumor cells between first and subsequent drain fluid samples is useful to monitor disease progression, diagnosis, chemotherapeutic efficacy, and may also provide insight into the biology of metastatic cancer. Tumor cells in the sample are quantified using any suitable detection technologies with or without enrichment, including but not limited to, fluorescence, surface-enhanced Raman scattering, or electrical impedance. The invention provides for assessing disease progression by identifying differences in an amount of tumor cells in first and subsequent drain fluid samples. Differences may be used to monitor disease progression, diagnosis, chemotherapeutic efficacy, and may also provide insight into the biology of metastatic cancer.

A common approach for CTC detection and isolation is immune-based detection, whereby antibodies are used to selectively bind cell surface antigens. Tumor cells express different cell surface markers than, for example, blood cells and therefore can be separated from the circulatory cells. Many CTC detection technologies have been developed and generally include capture, enrichment, detection, and release. The capture step is known as the specific interaction (such as physical interaction and antibody/antigen interaction) between CTCs and materials (e.g. magnetic beads, microfluidic chips). The enrichment step refers to isolation of CTCs from the blood. After enrichment, the CTCs could be detected by fluorescence (e.g. fluorescent microscope, fluorescent spectrophotometer and flow cytometry), surface-enhanced Raman scattering (SERS), electrical impedance, or any suitable method known to the person skilled in the art. The enriched CTCs can also be released for further phenotype identification and molecular analysis (e.g. mRNA profiling and cellular metabolism analysis). Shen, Zheyu et al. “Current detection technologies for circulating tumor cells.” Chemical Society reviews vol. 46, 8 (2017): 2038-2056. doi:10.1039/c6cs00803h

In another embodiment, disease progression is assessed by identifying a difference in the amount of a circulating, cell-free biomarker such as cell-free DNA (cfDNA) or cell-free RNA. The circulating, cell-free biomarker may also be extracellular vesicles, proteins, and metabolites from metastatic or normal organ physiologic turn over or impact of systemic drug treatment. For example, DNA methylation is an early event in cancer development that may be detected in circulating cell-free DNA. The information can be used for cancer diagnosis, prognosis, and monitoring.

cfDNA refers to all non-encapsulated DNA in blood. A portion of that cell-free DNA originates from a tumor clone and is called circulating tumor DNA (or ctDNA). cfDNA are nucleic acid fragments that enter the bloodstream during apoptosis or necrosis. Normally, these fragments are cleaned up by macrophages, but the overproduction of cells in cancer may leave more of the cfDNA behind. These fragments average around 170 bases in length, have a half-life of about two hours, and are present in both early and late-stage disease in many common tumors including non-small cell lung and breast. Additionally, circulating RNA is actively secreted by normal and cancer cells and can be found in biofluids together with other non-circulating or cell-free RNA.

Measurements obtained in methods of the invention may be quantified by any suitable analytical means In some embodiments cfDNA is quantified using QPCR using, for example, an Applied Biosystems Quantstudio 3D Digital PCR system. Methods for collection, extraction, fragment size determination, and concentration are known to a person skilled in the art as referenced in Chen, E., Cario, C. L., Leong, L. et al. Cell-free DNA concentration and fragment size as a biomarker for prostate cancer. Sci Rep 11, 5040 (2021). https://doi.org/10.1038/s41598-021-84507-z

Methods of the invention also include assessing disease progression by identifying differences in fragment size of a DNA or RNA species. For example, an average fragment size for a first fluid sample collected at the first time point is compared to an average fragment size obtained in a subsequent fluid sample collected at a later time. A difference in average fragment size is indicative of disease progression if there is a decrease in the average fragment size between the collection time points. The size profile may be assessed using any suitable analytical technique known to the person skilled in the art such as gel electrophoresis, atomic force microscopy, quantitative real-time PCR, or massively parallel sequencing.

The assessment of biomarkers between sample times may comprise determining an area under the curve (AUC) from a receiver operating characteristic (ROC) curve analysis. When a test is based on an observed variable that lies on a continuous or graded scale, an assessment of the overall value of the test can be made by using a receiver operating characteristic (ROC) curve. An ROC is a graphical plot that illustrates the diagnostic ability of a binary classifier system as its discrimination threshold is varied. The area under the receiver operating characteristic curve analysis is used to determine the discriminative ability of predictive models. For example, the AUC measures how well a parameter can distinguish between two diagnostic groups such as diseased and normal. The accuracy of a test is measured by the AUC, which can be calculated using any suitable computational and statistical software known to the person skilled in the art.

In alternative embodiments, the invention comprises methods for disease diagnosis in which a tumor cell or tumor-related nucleic acid is obtained from a surgical excision or biopsy. Then, a fluid sample is obtained from a lymphatic channel to determine whether the tumor cell or nucleic acid is present in the lymphatic channel fluid. Surgical procedures may include biopsy, excision, tumor resection, or other surgical interventions for treating, diagnosing, or staging disease. The invention provides for obtaining a surgical drain fluid sample from lymphatic channels during such surgery. The lymphatic channels are small thin blood vessels that do not carry blood, but rather collect and carry tissue fluid from the body to ultimately drain back into the blood. The lymphatic system consists of small lymphatic capillaries—termed initial lymphatics—that absorb interstitial fluid and cells to create lymph. These initial lymphatics bring lymph to the collecting lymphatic vessels, which are critical for transporting lymph over long distances through lymph nodes and eventually to the blood. The invention takes advantage of the recognition that the lymphatic system is also involved in cancer progression, as entry of metastatic cancer cells into the lymphatic system can result in lymph node metastases. Thus, the lymphatic system is central to a variety of pathological processes and many techniques have evolved to allow visualization of its anatomy and function

In some embodiments, methods of the invention include obtaining a fluid sample directly from the lymphatic channel by catheterization of the lymphatic channel. Sampling can be performed by any suitable procedure such as lymphatic cannulation as described in Lymphatic Cannulation for Lymph Sampling and Molecular Delivery, David C. Zawieja, et. al The Journal of Immunology Oct. 15, 2019, 203 (8) 2339-2350; DOI: 10.4049/jimmuno1.1900375, or otherwise known to a person skilled in the art.

Alternatively, the lymphatic channel can be visualized using radiographic methods or other suitable imaging methods such as magnetic resonance lymphography (MRL), positron emission tomography (PET), or near-infrared fluorescence imaging. Visualization of the lymphatic system can be used for a range of purposes such as diagnosing or treatment of disease, identifying and monitoring lymphedema, or for detecting metastatic lesions during cancer staging.

The invention is useful to predict metastatic disease by tracking the presence of a cancer-related biomarker from a locus of a tumor through the lymphatic channels to lymph nodes and finally to blood. The analysis can be halted at any point in the process. However, the rate of transit may provide significant diagnostic value, as well as value in therapeutic choice and efficacy. for example, the presence of a cancer biomarker in both the lymphatic channel and the lymph node indicates transit of cancer cells from tumor to lymph channel to lymph node. Further, identifying and tracking the movement of the cancer biomarker from the sample of lymphatic channel fluid, to lymph node, to blood can be used as a predictor of metastatic disease.

In some embodiments, the biomarker detected in a first sample is different than the biomarker detected in a second sample. In other preferred embodiments, the biomarkers detected across samples are of the same type. It is preferable to weight or quantify biomarkers in all samples taken in order to generate comparative analysis. Such comparative analysis is indicative of the rate of progress of disease and its severity. In one embodiment, the invention comprises identifying a cancer biomarker in the lymphatic channel fluid sample and determining whether the same cancer biomarker is present in a lymph node. In addition to a presence/absence test, the amount of the cancer biomarker in the lymphatic channel fluid can be quantified and compared to a quantifiable amount of the cancer biomarker in the lymph node. The cancer biomarker may be selected from any suitable biomarker including a nucleic acid, a protein, or a tumor cell as described above. In one embodiment, the cancer biomarker is selected from a nucleic acid, a protein, and a tumor cell. The cancer biomarker may also be a ratio of circulating tumor cells to cell-free DNA. Quantification may be performed by any suitable method including those described above and known to the person with skill in the art.

Identifying the biomarker in the lymphatic channel fluid and determining if the same cancer biomarker is in a lymph node may take place at the same time point. The method also provides that identifying a cancer biomarker in lymphatic channel fluid and determining whether the same cancer biomarker is in a lymph node may take place at two or more different time points.

The invention may further comprise analyzing a blood sample for the same biomarker. In one embodiment, the cancer biomarker is identified in blood prior to identifying the cancer biomarker in lymphatic channel fluid. For example, if the biomarker is found in the drain fluid sample but not in the blood, then, although the disease has moved from the tumor into the lymphatic channel, it has not moved to the blood. Identifying and movement of the cancer biomarker from the sample of lymphatic channel fluid, to lymph node, to blood can be used as a predictor of metastatic disease.

The invention discloses a method for assessing cancer metastasis. Contemplated methods comprise identifying circulating tumor cells in lymphatic fluid at two or more time points; identifying cell-free tumor DNA in lymphatic fluid at two or more time points; comparing a ratio of circulating tumor cells to cell-free DNA at the same two or more time points; and assessing the risk of metastasis as a changing ratio of cell-free tumor DNA to circulating tumor cells over the time interval of the two time points.

In some embodiments, methods of the invention provide for obtaining a surgical drain fluid sample during a surgery. In one embodiment, the lymphatic fluid is separated from the drain fluid and analyzed for circulating tumor cells and cell-free tumor DNA. Separation of lymphatic fluid may be accomplished by any suitable separation process, such as filtration or gravimetric separation. The lymphatic fluid may also be obtained directly from a surgical site as described above or by using any suitable method known to a person skilled in the art.

The invention includes methods for disease staging. Staging is the process of determining the extent of cancer within a patient's body, where it is located, and whether the disease has spread from where it first formed to other parts of the body.

Preferred methods comprise the steps of obtaining DNA from a sample of tumor tissue; identifying cell-free tumor DNA in lymphatic fluid; identifying cell-free tumor DNA in blood; and assessing disease stage based on ratios of tumor DNA in the tumor, the lymphatic fluid, and the blood. The sample of tumor tissue may be obtained from a biopsy, excision, or resection. The sample of lymphatic fluid may be obtained from surgical drain wasted during a surgery or directly from the surgical site as described above. The tumor tissue sample, the lymphatic fluid sample, and the blood sample may be taken at the same or different time points. The cell-free DNA from the three samples is quantified using any suitable analytical quantifying technique as described above and known to a person skilled in the art. The ratios of the tumor DNA in the tumor, lymphatic fluid, and blood are an indicator of disease staging.

Tumor DNA may be obtained using any suitable method such as touch imprint cytology (TIC) to obtain genomic DNA from cancer cells, which can be observed under a microscope. Cell morphology and cancer cell numbers can be evaluated using TIC specimens as described in Amemiya, Kenji et al. “Simple and Rapid Method to Obtain High-quality Tumor DNA from Clinical-pathological Specimens Using Touch Imprint Cytology.” Journal of visualized experiments: JoVE, 133 56943. 21 Mar. 2018, doi:10.3791/56943. Alternatively, a commercially available test kit such as the ThermoFisher ChargeSwitch gDNA Mini and Micro Tissue Kits, which allow for rapid and efficient purification of genomic DNA from mini (10-25 mg) or micro (3-5 mg) quantities of tissue, respectively, may be used.

This disclosure provides methods for molecular staging of cancer. The elimination of metastases remains one of the major challenges in the curative treatment of patients with cancer. Recently developed molecular staging approaches, including coupling disease-specific markers with a powerful detection technology like quantitative reverse transcriptase-polymerase chain reaction (qRT-PCR), offer a sensitive detection system for metastases as described in Mejia, Alex et al. “Molecular staging individualizing cancer management.” Journal of surgical oncology vol. 105,5 (2012): 468-74. doi:10.1002/jso.21858, and known to a person skilled in the art.

Methods for molecular staging include the steps of determining an amount of cell-free tumor DNA in lymphatic fluid; determining an amount of cell-free DNA in blood; and assessing disease stage based on the relative amounts of the cell-free tumor DNA in both the lymphatic fluid and in the blood. The lymphatic fluid sample may be obtained from surgical drain waste during a surgery as described above. The amount of cell-free tumor DNA in both the lymphatic fluid sample and the blood sample is quantified using any suitable quantifying analytical method as described above and known to a person skilled in the art. Additionally, the length of the cell-free tumor DNA may be determined. The method is also used to determine the rate of transit of the cell-free DNA from the lymphatic fluid to the blood.

The invention provides methods for assessing metastatic disease. The methods comprise the steps of identifying a cancer biomarker in at least two of lymphatic channel fluid, lymph node tissue, and blood; and assessing metastatic disease based on relative amounts of the biomarker in the at least two samples used. The lymphatic channel fluid may be obtained from surgical drain waste during a surgery, for example surgery for lymph node biopsy or lymphadenectomy. Preferred methods use samples from at least two of lymphatic channel fluid, lymph node tissue, and/or blood. The biomarker used may be any suitable biomarker for assessing disease as described above. The amount of biomarker in each of the at least two kinds of samples analyzed is quantified and compared using any commercially available method and as described above. Metastatic disease is assessed by comparing the relative amounts of the biomarker in the lymph node tissue sample, the lymphatic channel fluid sample and/or blood sample. The quantified amount relative amount indicates the transit of tumor cells from tumor, to lymphatic channel, to lymph node to blood. This indication is used to assess the metastasis of the disease.

The invention includes methods of assessing disease wherein the method comprises the steps of accessing fluid that exists between a tumor and a lymphatic channel; accessing the fluid that exists between a lymphatic channel and a lymph node; or accessing the fluid that exists between a lymph node and blood. Accessing the fluid and sampling the fluid can be performed using any suitable method as described above or as known to a person skilled in the art. The method further comprises the step of performing an assay on the fluid to detect molecular indicia of disease.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for therapeutic selection, the method comprising
obtaining a drain fluid proximal to a tumor;
evaluating a biomarker indicative of a therapeutic efficacy against the tumor; and
selecting a targeted therapy based on the evaluating step.

2. The method of claim 1, wherein the drain fluid is selected from blood, urine, saliva, cerebrospinal fluid, sputum, interstitial fluid, pleural fluid, bronchioalveolar lavage, and lymph.

3. A method for therapeutic screening, the method comprising
obtaining a surgical drain fluid sample and a tumor biopsy sample;
evaluating one or more biomarkers in the surgical drain fluid sample;
selecting a putative therapeutic based on the evaluating step;
applying the therapeutic to the tumor biopsy sample; and
determining an effect of the therapeutic on the tumor biopsy sample.

4. The method of claim 3, wherein said method is multiplexed across a plurality of tumor biopsy samples simultaneously.

5. The method of claim 3, wherein the tumor biopsy sample is cultured prior to the applying step.

6. A method for screening patients for efficacy of anti-cancer therapy, the method comprising
obtaining a sample of drain fluid proximal to a tumor;
evaluating a biomarker indicative of a response to therapy;
providing a selected therapy based on said biomarker;
obtaining a second body fluid sample; and
evaluating efficacy of the therapy based on a comparison of the first and second samples.
